(19) United States
(12) Patent Application Publication
(10) Pub. No.: US 2018/0368934 A1
(43) Pub. Date: Dec. 27, 2018

(54) ELONGATED INTERVENTIONAL DEVICE FOR OPTICAL SHAPE SENSING

(71) Applicant: KONINKLIJKE PHILIPS N.V., Eindhoven (NL)

(72) Inventors: Franciscus Reinier Antonius VAN DER LINDE, Schundel (NL); David Paul NOONAN, New York, NY (US); Marcellinus Petrus Maria CNOOPS, Breda (NL); Bout MARCELIS, Eindhoven (NL)

(73) Assignee: Koninklijke Philips N.V., Eindhoven (NL)

(21) Appl. No.: 16/064,018

(22) PCT Filed: Dec. 27, 2016
(86) PCT No.: PCT/EP2016/082721

§ 371 Date: Jun. 20, 2018

Related U.S. Application Data
(60) Provisional application No. 62/271,417, filed on Dec. 28, 2015.

(30) Foreign Application Priority Data
Mar. 3, 2016 (EP) 16158519.5

(51) Int. Cl. A61B 90/00; A61M 25/00

(52) U.S. Cl. CPC A61B 90/37; A61B 2034/2061; A61M 25/0054; A61M 25/0053

(57) ABSTRACT

The invention relates to an elongated interventional device (1), configured to receive an optical shape sensing fiber (9) and comprising (i) an elongated proximal section (22) including at fixation element (24) at its proximal end, the fixation element (24) being connectable to a reception (25) at a predetermined location (3); and (ii) an elongated distal section (21) connected to the proximal section (22). The proximal section (22) has a lower torsion stiffness than the distal section (21) and comprises at least two substantially co-axial tubes, the outer tube having a lower torsion stiffness than an inner tube. Thus, the elongated interventional device (1) can be affixed to the predetermined location in order to determine its shape and position by means of optical shape sensing without affecting the device's maneuverability, particularly with respect to rotational movements of the distal section (21).

### FIELD OF THE INVENTION

The invention relates to an elongated interventional device that can be located by optical shape sensing and to an interventional system including the elongated interventional device.

### BACKGROUND OF THE INVENTION

In certain interventional procedures, a physician may direct elongated interventional devices, such as catheters or guide wires, to target locations within a patient's body, wherein the positions of the elongated interventional devices may be determined by optical shape sensing. In optical shape sensing, an optical fiber may be integrated into the elongated interventional device and the light reflected from stain sensors included in the optical fiber is measured in order to determine the shape of the optical fiber and, thus, the shape of the elongated interventional device.

On the basis of the shape of the elongated interventional device, the position of the interventional device may be determined in a certain reference frame. For this purpose, a proximal end section of the optical fiber and, thus, the elongated interventional device, may be affixed to a defined location in the reference frame. However, such a fixation of the elongated interventional device limits its maneuverability, particularly with respect to rotational motions of the elongated interventional device around its longitudinal extension. Due to the fixation, torsional stress builds up within the elongated interventional device and such torsional stress makes rotational motions difficult and makes it difficult to keep the device in a desired (rotational) position.

EP 0 369 383 A2 discloses a flexible catheter, which comprises a resilient flexible tubular layer in telescoping relation and bonded to a tubular wire sheath. By variation of the strand angle in the wire sheath, varying sections of the catheter with different torsional and longitudinal rigidity can be accomplished. Moreover, the strands of the wire sheath adjacent to the proximal end of the catheter are parallel to the axis of the catheter. This provides a section with a high longitudinal stiffness and a low torsional stiffness. Further, an outer plastic layer may be applied over the wire sheath.

### SUMMARY OF THE INVENTION

It is an object of the present invention to improve the maneuverability of an elongated interventional device particularly with respect to rotational motions, when a proximal end section of the elongated interventional device is fixated in order to locate the device by means of optical shape sensing.

In one aspect of the present invention, an elongated interventional device is suggested. The elongated interventional device is configured to receive an optical shape sensing fiber and comprises an elongated proximal section including at fixation element at its proximal end. The fixation is being connectable to a reception at a predetermined location. Further, the elongated interventional device comprises an elongated distal section connected to the proximal section. The proximal section has a lower torsion stiffness than the distal section and comprises at least two substantially co-axial tubes, the outer tube having a lower torsion stiffness than an inner tube.

Since the proximal section of the elongated interventional has a lower torsion stiffness than the distal section, torque applied to the distal section can be absorbed by the proximal section. Thus, the distal section of the elongated interventional device can be decoupled from the fixation of the device at the predetermined location with respect to rotational movements of the distal section. Hereby, the maneuverability of the elongated interventional device can be maintained despite its fixation at the predetermined location.

The term torsion stiffness particularly relates to the resistance against an applied torque or torsion moment and a higher torsion stiffness corresponds to a higher resistance against an applied torque.

Further, the proximal section comprises at least two substantially co-axial tubes, and an outer tube has a lower torsion stiffness than an inner tube. In accordance with this configuration, the reduced torsion stiffness of the outer tube, which provides the largest contribution to the torsion stiffness of the proximal section of the elongated interventional device, ensures the torque absorbing characteristic of the proximal section of the device.

In one embodiment of the invention, the proximal section and/or the distal section have a substantially homogenous torsion stiffness. With respect to the proximal section, a homogenous (lower) torsion stiffness ensures that the proximal section can absorb torque substantially along its entire longitudinal extension. With respect to the distal section, a homogenous (higher) torsion stiffness ensures that a rotation of the proximal end of the distal section is transferred to the distal end of the distal section as usually desired in order to facilitate the handling of the device.

In one embodiment, the inner tube has a higher kink resistance than the outer tube. In this embodiment, the inner tube can ensure a sufficiently high kink resistance of the proximal section of the elongated interventional device. The term kink resistance relates to the resistance against kinking and a higher kink resistance corresponds to the higher resistance against kinking. Thus, it is particularly possible to avoid kinks or pitch points of the optical shape sensing fiber integrated into the elongated interventional device, which would affect the determination of the shape and position of the elongated interventional device by optical shape sensing. In particular, the kink resistance may be parameterized on the basis of an inverse of the minimum bending radius, which can be employed to a section of the device without producing a kink, or an inverse of the bending radius which just produces a kink.

One related embodiment of the invention includes that only the inner tube comprises at least one metal wire. A further related embodiment includes that a plurality of metal wires form a braiding and/or wherein at least one metal wire forms a spiral. By means of the one or more metal wires included in the inner tube, the desired kink resistance of the inner tube can be achieved.

In a further embodiment, the proximal section comprises three substantially co-axial tubes and an innermost tube has a lower friction coefficient than the other tubes. This particularly means that there is a lower force of friction between the surface, particularly the inner surface, of the innermost tube and the surface of another material than between the surface of the other tubes and the same material, when the same normal force is applied to the surface. In this embodiment, the innermost tube may receive the optical shape sensing fiber, which may be manually inserted into this tube, or it may receive another means manually inserted into the tube. The lower friction coefficient of the tube facilitates the insertion of the optical shape sensing fiber or the other means.

A further embodiment of the invention includes that the fixation element is configured to affix only the outer tube to the reception. In a related embodiment, the inner tube can move relative to the outer tube of the proximal section. In this embodiment, the transfer of torque from the outer tube to the inner tube having a higher torsion stiffness is minimized so that substantially the complete torque is absorbed by the outer tube affixed to the reception. Hereby, the torsion stiffness of the proximal section of the elongated interventional device can be further reduced.

The elongated interventional device according to the invention can be configured in different ways depending on the desired purpose of the device. In some embodiments, the elongated interventional device particularly comprises a catheter or guide wire. Likewise, the elongated interventional device may comprise an endoscope, for example.

In a further aspect of the invention, an interventional system for performing an interventional procedure at a patient body is suggested. The interventional system comprises an elongated interventional device as described above including the optical shape sensing fiber. Further the system comprises the reception arranged at the predetermined location and connectable with the fixation element of the elongated interventional device. Moreover, the interventional system comprises an optical shape sensing device, which is coupled to the optical shape sensing fiber and which is configured to determine a shape of the optical fiber and a position of the optical fiber relative to the predetermined location by optical shape sensing.

In one embodiment, the optical shape sensing device is configured to generate an image of the elongated interventional device on the basis of the determined shape and position. In a related embodiment, the optical shape sensing device is configured to overlay the image of the elongated interventional device and an image of the patient body, the image of the patient body being shown in accordance with a relative position of the patient body with respect to the predetermined location. In particular, the optical shape sensing device may configured to overlay the images in such a way that the relative position of the image of the elongated interventional device and the image of the patient body correspond to the relative position of the interventional device and the patient body.

When the images of the elongated interventional device and the patient body are overlaid in such a way, a physician can particularly monitor the position of the elongated interventional device within the patient body during the interventional procedure. Such an overlaying is made possible since the elongated interventional device or the proximal end thereof is affixed to the predetermined location so that its position relative to the patient body can be determined (when the relative position of the patient and the predetermined location is known).

The image of the elongated interventional device and/or the image of the patient body may be three-dimensional images. Moreover, the image of the patient body preferably shows the inner of the patient body. Such an image may be acquired using a suitable imaging technique known to a person skilled in the art, such as computed tomography (CT) imaging or magnetic resonance imaging (MRI).

It shall be understood that the elongated interventional device of claim 1 and the interventional system of claim 10 have similar and/or identical preferred embodiments, in particular, as defined in the dependent claims.

It shall be understood that a preferred embodiment of the present invention can also be any combination of the dependent claims or above embodiments with the respective independent claim.

These and other aspects of the invention will be apparent from and elucidated with reference to the embodiments described hereinafter.

### BRIEF DESCRIPTION OF THE DRAWINGS

In the following drawings:

FIG. 1 schematically and exemplarily shows an interventional system comprising an elongated interventional device,

FIG. 2 schematically and exemplarily shows an elongated interventional device in one embodiment, and

FIG. 3 schematically and exemplarily shows a cross section of a proximal section of the elongated interventional device in one embodiment.

### DETAILED DESCRIPTION OF EMBODIMENTS

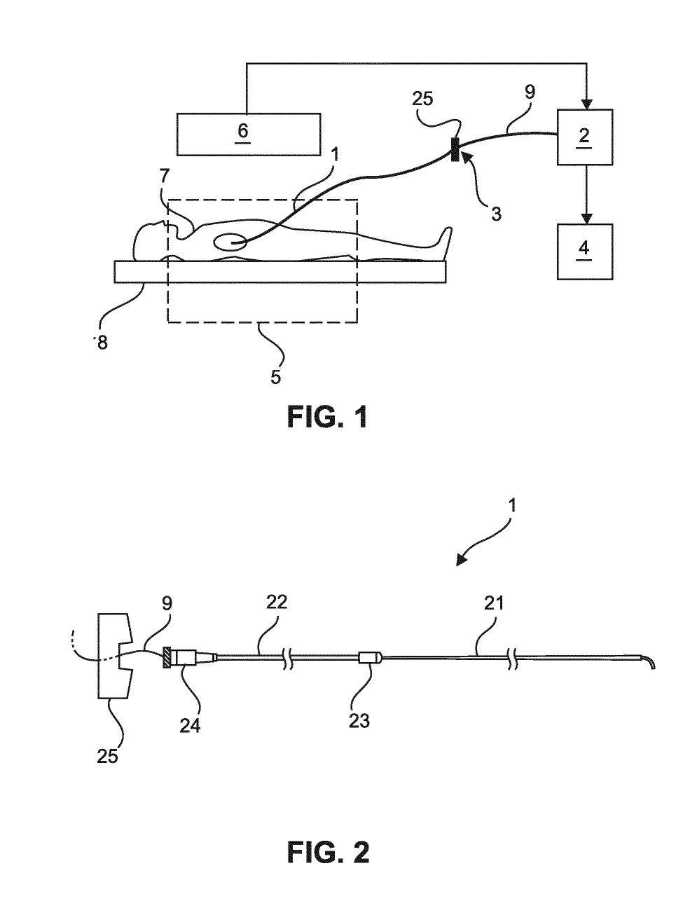

FIG. 1 exemplarily and schematically shows an interventional system comprising an elongated interventional device 1. The elongated interventional device may be configured as a catheter, a guide wire, an endoscope or a similar device that a physician inserts into a patient's body 7 during an interventional procedure, when the patient's body 7 is positioned in an examination region 5. In the examination region 5, the patient's body may be positioned on a patient table 8 or a similar support.

For determining the shape of the elongated interventional device 1 during the interventional procedure, an optical shape sensing technique is applied. Thus, an optical shape sensing fiber 9 is integrated into the elongated interventional device 1 such that the optical fiber 9 preferably extends along the longitudinal extension of the elongated interventional device 1, preferably but not necessarily over the complete length of the elongated interventional device 1. The optical shape sensing fiber 9 can be coupled to an optical shape sensing device 2 which may determine the shape of the optical shape sensing fiber 9 using any known shape sensing technique. In particular, the optical fiber 9 may be provided with strain-sensitive fiber Bragg grating (FBG) sensors, and the optical shape sensing device 2 may inject light into the optical shape sensing fiber 9 and my determine the shape of the optical shape sensing fiber 9 from light reflected by the FBG sensors. One example of a suitable technique for determining the shape of the optical fiber 9 in such a way is disclosed in WO 2013/136247 A1, which is herewith incorporated by reference.

Moreover, the position of the elongated interventional device 1 is determined relative to a fixed predetermined location 3. For this purpose, the proximal end of the elongated interventional device 1 is fixated at the predetermined location 3. As a consequence, the location of the proximal end of the elongated interventional device 1 is known and corresponds to the predetermined location 3. The position of any other point or section of the elongated interventional device 1 can be determined in the optical shape sensing device 2 on the basis of the determined shape of the optical shape sensing fiber 9. Thus, the optical shape sensing device 2 can determine the position of the entire elongated interventional device 1 with respect to the predetermined location 3.

The determined shape and position of the entire elongated interventional device 1 or at least the shape and position of a distal section of the device 1 including the portion of the device that is inserted into the patient's body 7 is preferably shown on a display device 4 connected to the optical shape sensing device 2. For this purpose, the optical shape sensing device 2 generates an image of the elongated interventional device 1 which is shown on the display device 4 and which is dynamically adapted in accordance with the shape and position of the optical shape sensing fiber 9, which may be determined quasi-continuously (i.e. in small time intervals). Thus, the physician using the elongated interventional device 1 during an interventional procedure can monitor the position of the device including the position of the portion of the device 1 that is inserted into the patient's body 7 and, thus, cannot be viewed.

In one implementation, the optical shape sensing device 2 additionally controls the display device 4 to show a three-dimensional image of the inner of the patient's body 7 and overlays the three-dimensional image of the inner of the patient's body 7 with the image of the elongated interventional device 1. The overlaying is made on the basis of the determined position of the elongated interventional device 1 and is particularly made in such a way that the relative position of the images of the patient's body 7 and of the elongated interventional device 1 correspond to the relative position of the patient's body 7 and the device 1. In order to overlay the images of the patient body 7 and the elongated interventional device 1 in such a way, the patient is positioned in the examination region 5 such that the body portion shown in the image has a defined relative position with respect to the predetermined location 3.

The three-dimensional image of the patient body 7 can be acquired using any suitable imaging modality known to a person skilled in the art. Examples of such imaging modalities include computed tomography (CT) imaging and magnetic resonance imaging (MRI), and the image may be acquired using an imaging device 6 configured in accordance with the employed imaging modality.

In one embodiment, the imaging device 6 is integrated into the interventional system including the elongated interventional device 1. In this embodiment, the imaging device 6 acquires three-dimensional images with respect to a field of view including at least a part of the examination region 5. The field of view has a defined relative position with respect to the predetermined location 3 so that the shape sensing device 2 can overlay the images generated using the imaging device 6 and the images of the elongated interventional device 1 in the way described above. Moreover, the imaging device 6 may quasi-continuously acquire images in certain time intervals during an interventional procedure, and, at each point in time, the shape sensing device 2 may overlay the current image of the elongated interventional device 1 and the most recently acquired three-dimensional image of the patient's body 7. Thus, the physician can monitor the position of the elongated interventional device in the current environment with the patient's body 7 substantially in real-time.

As an alternative, the three-dimensional image of the patient's body 7 may be acquired using an external imaging device that is not integrated into the interventional system. By means of the external imaging device the image of the patient's may be acquired prior to the interventional procedure and transmitted to the shape sensing device 2. For carrying out the interventional procedure, the patient may be positioned in such a way that the portion of the patient's body 7 shown in the image has a defined relation position with respect to the predetermined location 3. This allows for overlaying the three-dimensional image of the patient's body 7 and the image of the elongated interventional device 1 as described above.

An embodiment of the elongated interventional device 1 is schematically and exemplarily shown in FIG. 2. As shown in the figure, the device 1 comprises a distal section 21 and a proximal section 22.

In the embodiment shown in FIG. 2, the distal section 21 and the proximal section 22 are connected to each other by means of a transition element 23. For this purpose, both the distal section 21 and the proximal section 22 are affixed to the transition element 23 by means of a suitable connection. For instance, the distal section 21 and the proximal section 22 may glued together with the transition element 23, respectively, or the relevant ends of the distal section 21 and the proximal section 22 may be molded into the transition element 23. Likewise, it is possible to affix the distal section 21 and the proximal section 22 to the transition element 23 by means of a bolted connection. The transition element 23 is particularly configured in such a way that tubes and/or lumina of the proximal section 22 are connected to corresponding tubes and/or lumina of the distal section of the elongated interventional device 1.

As an alternative, the distal section 21 and the proximal section 22 of the elongated interventional device 1 may be directly connected to each other. This does particularly mean that tubes and/or lumina of the proximal section 22 are directly connected to corresponding tubes and/or lumina of the distal section of the elongated interventional device. The connection can be established by any suitable means which may be selected based on the materials included in the distal section 21 and the proximal section 22. In particular, the distal section 21 and the proximal section 22 may be thermally bonded or glued together. In case the distal section 21 and the proximal section 22 include metal as explained further below, they may also be soldered together.

In use, the physician may particularly hold the elongated interventional device 1 at the distal section 21 thereof, particular at a proximal end portion of the distal section 21. The distal section 21 comprises the part of the elongated interventional device 1 which is (potentially) inserted into the patient's body 7 during the interventional procedure. The distal section 21 may be configured in the same way as the corresponding section of a conventional elongated interventional device of the relevant type. If the elongated interventional device 1 is a catheter, it may e.g. comprise one tube or plural co-axial tubes made of a suitable material and having a suitably formed end portion. If the elongated interventional device 1 is a guide wire, it may comprise one tube or plural co-axial tubes and a guide wire core, for example, each component being made of a suitable material.

Further, the optical shape sensing fiber 9 is integrated into the distal section 1 in a suitable way. For instance, if the distal section 21 comprises one tube or plural co-axial tubes, the optical shape sensing fiber 9 may be guided within the tube or within the inner one of the co-axial tubes. As an alternative, an additional lumen may be provided for receiving the optical shape sensing fiber 9. In case the distal section 21 comprises plural co-axial tubes, this lumen may particularly be provided between the outer surface of an inner tube and the inner surface of an outer tube

As with conventional elongated interventional devices, the materials of the components of the distal section 21 are selected such that the distal section 1 has the desired properties. These properties typically include a sufficient kink resistance, which particularly prevents interruptions of the light beam traveling through the optical shape sensing fiber 9. When the kink resistance is higher, a higher force has to be applied to the device to produce a kink. One parameter, which may be used for quantifying the kink resistance may be a parameter corresponding to and/or being derived from the inverse of the minimum bending radius, which can be employed to a section of the device without producing a kink, or the inverse of the being radius which just produces a kink. Moreover, the distal section 21 preferably has a high torque stiffness so that also subtle rotational movements at the proximal end of the distal section 21 are transmitted directly to the distal end of the distal section 21. This ensures a good maneuverability of the elongated interventional device 1 during the interventional procedure.

The (proximal) end of the proximal section 22 is fixated at the predetermined location 3. The fixation can in principle be realized in any suitable way. In one embodiment, a fixation element 24 is provided at the proximal end of the proximal section 22, which is configured such that it can be connected to a corresponding reception 25 of the interventional system, which is arranged at the predetermined location 3. In principle, the fixation element 24 and the reception 25 may be configured in any suitable way known to a person skilled in the art. In one embodiment, the reception 25 may be configured as a socket and the fixation element 24 may be configured as a plug, which can be inserted into the socket and is held in place within the socket by suitable means. In a further configuration, the fixation element 24 and the reception 25 may be connectable to each other by means of a screw coupling. For this purpose, the fixation element 24 may comprise a male screw that can be connected to a female screw of the reception 25 or vice versa.

Via the reception 25, the optical shape sensing fiber 9 integrated into the elongated interventional device 1 is preferably also coupled to the optical shape sensing device 2. Moreover, components of the elongated medical device 1 may be connected to further devices of the interventional system. For instance, the elongated interventional device 1 or a tube included therein may be connected to a suction device in case the elongated interventional device 1 is configured as a suction catheter. In further embodiments, the interventional device 1 may be connected to components of a stent delivery system, if the elongated interventional device 1 is used for inserting stents into patient's bodies 7, or it may be connected to a pump, if the interventional device 1 is used as a balloon catheter, for example.

Due to the fixation of the proximal end of the elongated interventional device 1 at the predetermined location, the maneuverability of the device 1 can potentially be restricted. In particular, the fixation could make rotational movements of the device 1 more difficult, and could make it more difficult to hold the device 1 in place after torsional stress has built up.

In order to avoid such restrictions of the maneuverability of the elongated interventional device 1, the proximal section 22 of the elongated interventional device 1 is configured such that it has a low torsion stiffness, particularly a lower torsion stiffness than the distal section 21 of the elongated interventional device 1. The torsion stiffness corresponds to a parameter quantifying the resistance against an applied torque and a higher torsion stiffness corresponds to a higher resistance against an applied torque. Thus, the proximal section of the elongated interventional device has a lower resistance against an applied torque compared with the distal section of the device. Hereby, torque applied to the distal section 21 of the elongated interventional device 1 is absorbed in the proximal section 22 so that the proximal section 22 applies no or a very low torque to the distal section 21. In particular, the torsion stiffness may be parameterized by the ratio of the torque and the twist angle, i.e. by k=M/θ where k is the torsion stiffness, M is the applied torque and θ is the resulting angle of twist. Here, the angle of twist particularly relates to the relative angle of rotation between one end of a body and the opposite end with respect to the rotation axis, which results from the applied torque.

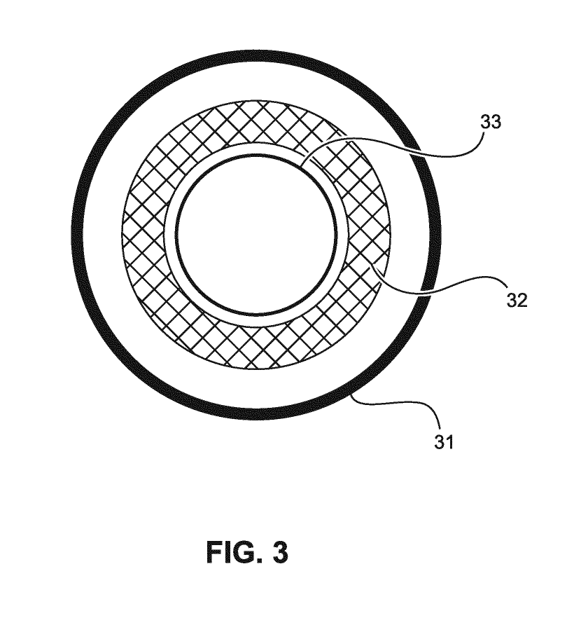

In some embodiment, the proximal section 22 of the elongated interventional device 1 comprises two or three concentric tubes. These embodiments are schematically and exemplarily illustrated in FIG. 3 showing a cross section of the proximal section 22 of the device 1 with an outer tube 31 and a first inner tube 32. Optionally, an additional second inner tube 33 is provided within the first inner tube 32. The inner tube(s) 32, 33 of the proximal section 22 may be connected to one or more corresponding tube(s) of the distal section 21 of the elongated interventional device 1 by means of a direct connection or a transition element 23 as explained above. The outer tube 31 may likewise be connected to a corresponding tube of the distal section 21. Likewise, it is possible that the distal section 21 does not have a tube corresponding to the outer tube 31 of the proximal section 22 and that the outer tube 31 is only connected to the transition element 23.

In one embodiment, the optical shape sensing fiber 9 is guided through the innermost tube 32, 33 of the proximal section 22. This innermost tube 32, 33 may be connected with the tube or lumen for receiving the optical shape sensing fiber 9 in the distal section 21 of the elongated interventional device 1, or the innermost tube 32, 33 of the proximal section 22 may extend into the distal section 21 such that it also forms the tube for receiving the optical shape sensing fiber 9 in the distal section 21. Thus, the optical shape sensing fiber 9 can extend along the complete length of the elongated interventional device 1.

The optical shape sensing fiber 9 may be fixedly integrated in the elongated interventional device 1, or it may be manually inserted into the elongated interventional device 1 when needed. In the latter case, the optical shape sensing fiber 9 may be inserted into the elongated interventional device 1 from the proximal end thereof. In order to facilitate such an insertion, the proximal section 22 of the interventional device 1 is preferably provided with the aforementioned second inner tube 33 which receives the optical shape sensing fiber 9 and which may be made of a material having a low friction coefficient. In further embodiments, the optical shape sensing fiber 9 may be guided through a further dedicated lumen of the proximal section 22 of the elongated interventional device 1. This lumen may be connected to a corresponding lumen for receiving the optical shape sensing 9 in the distal section 21, or the lumen may extend into the distal section. In case the proximal section 22 comprises plural co-axial tubes, the lumen may be arranged between two tubes, for example.

The desired low torsion stiffness of the proximal section 22 of the elongated interventional device 1 is particularly ensured by the outer tube 31, since the outer tube 31 has the largest diameter and provides the largest contribution to the torsion stiffness of the proximal section 22. Thus, the outer tube 31 is made of a suitable material which ensures that the outer tube 31 has a lower torsion stiffness than the distal section 21 of the elongated interventional device 1. The inner tubes 31 and 32 may ensure further desired characteristics of the proximal section 22 of the device 1, such as kink resistance. This may require that at least one inner tube 32 or 33 is made of material which results in a higher torsion stiffness. Therefore, the outer tube 31 may also have a lower torsion stiffness than one or both of the inner tubes 32 and 33.

In order to ensure that the outer tube 31 has a low torsion stiffness, it is made of a flexible material, such as a rubber or polymer material, and preferably, the outer tube 31 does not include a metal braiding or coiling (unlike one of the inner tubes 32, 33 in one embodiment). Suitable materials for manufacturing the outer tube 31 include: Polyether block amide (PEBA), which is also known as Pebax; Nylon; polyurethane; polytetrafluoroethylene (PTFE); polyethylene terephthalate (PET); polyether ether ketone (PEEK); and polyethylene (PE), particularly high-density polyethylene (HDPE), low-density polyethylene (LDPE) and high-molecular-weight polyethylene (HMWPE).

As explained above, the inner tube 32 particularly ensures a sufficient kink resistance of the proximal section 22 of the elongated interventional device 1. Hereby, kinks or pinch points of the optical shape sensing fiber 9 can particularly be avoided. In order to achieve a sufficient kink resistance, the inner tube 32 may include metal. In particular, the inner tube 32 may include one or more metal wires which are arranged in a coiling or braiding. Hereby, it is also possible to minimize elongation of the proximal section 22 when torsional stress is applied thereto. In addition, the inner tube 32 may also include a rubber or polymer material, and the coiling or braiding may be integrated into the rubber or polymer material or the inner tube 32 may include plural layers, where one layer may include the rubber or polymer material and another layer may include the metal coiling or braiding. When the inner tube includes a rubber or polymer material, there may be a certain larger spacing between the metal wires of the coiling or braiding. Hereby, the bending stiffness of the proximal section 22 can be reduced so that its flexibility and, thus, its maneuverability are improved. The bending stiffness corresponds to the resistance of the tube against bending deformation. It may particularly be quantified by means of the ratio between an applied force and the resulting deflection of the tube.

In some embodiments, the proximal section 22 of the elongated interventional device 1 includes aramid fibers, preferably instead of metal wires. These fibers may be integrated into the outer tube 31 or the inner tube 32. As an alternative, a tube-shaped braiding or webbing of aramid fibers may also be included into the proximal section 22 between the outer tube 31 and the inner tube 32. Due to their high tensile strength and flexibility, such aramid fibers minimize the elongation of the proximal section 22 of the elongated interventional device 1 ensure a low bending stiffness.

Optionally, the additional inner tube 33 is provided within the first inner tube 32 as explained above. In particular, the additional inner tube 33 can be provided in case the optical shape sensing fiber 9 is manually inserted into the elongated interventional device 9. Likewise, the additional inner tube 33 may be provided when the intended use of the elongated interventional device 1 comprises a (manual) insertion of other means, such as a wire or a (further) catheter, into the elongated interventional device 1. In order to facilitate the insertion of the optical shape sensing fiber 9 or the other means, the additional inner tube 33 is made from a material having a low friction coefficient, particularly a lower friction coefficient than the other tubes. The friction coefficient particularly corresponds to ratio between the force of kinetic friction between the (inner) surface of the tube 33 (or another tube of the elongated interventional device 1) and the surface of another material and the normal force for pressing the surfaces together. More specifically, the friction coefficient may correspond to an average friction coefficient of this ratio with respect to other materials which typically come into contact with the tube 33 (or the other tube) in the use of the elongated interventional device, such as the materials of the optical shape sensing fiber 9 and/or the aforementioned other means.

In order to achieve the desired low friction coefficient, the additional inner tube 33 may be made of a suitable material, such as polytetrafluoroethylene (PTFE), polyimide (PI), a combination of PI and PTFE, or high-density polyethylene (HDPE). Moreover, the additional inner tube 33 preferably has a thinner wall than the first inner tube 32. Hereby, the space required for the additional inner tube 33 within the elongated interventional device 1 is minimized and the contribution of the additional inner tube 33 to the mechanical properties of the elongated interventional device 1 is reduced. Preferably, the additional inner tube 33 and the inner tube 32 are bonded together. Hereby, a smooth transition between these tubes is ensured.

When the proximal section 22 of the elongated interventional device 1 comprises an outer tube 31 and one or more inner tubes 32, 33, preferably only the outer tube 31 is fixated at the predetermined location 3. Thus, only the outer tube 31 is affixed to the fixation element 24. The inner tube 32 may be in contact with the outer tube 31 but is preferably not bonded to the outer tube 31 so that the inner tube 32 and outer tube 31 can move and particularly rotate relative to each other. When torque is applied to the proximal section 22 of the elongated interventional device 1, the outer tube 31 may thus move (twist) and the inner tube 32 may not or only minimally follow such movement of the outer tube 31. Hereby, the torsion stiffness of the proximal section 22 of the device 1 can be further reduced.

In the aforementioned embodiments of the proximal section 22 of the elongated interventional device 1, the desired characteristics of the proximal section 22 such as low torsion stiffness and high kink resistance are achieved by means of plural co-axial tubes, where the outer tube 31 is configured such that a low torsion stiffness is achieved and where an inner tube 32 is configured such that a high kink resistance is achieved.

In further embodiments, the proximal section 22 of the elongated interventional device 1 consists of a single tube, to which the fixation element 24 and the transition element 23 are affixed. In order to ensure a sufficient kink resistance, the tube may include a metal or aramid reinforcement such as a coiling or braiding formed of one or more metal wires or aramid fibers. The use of a coiling is preferred in order to ensure the desired low torsion stiffness and a low bending stiffness. If a braiding is provided, the braiding preferably has a small braiding angle in order to ensure the desired low torsion stiffness. Further, the diameter of the tube and its wall thickness are suitably selected in order to ensure the desired properties such as a low torsion stiffness, a low bending stiffness and a high kink resistance. The optical shape sensing fiber 9 may be inserted in a lumen provided with the tube for receiving the shape sensing fiber 9.

Variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality.

A single unit or device may fulfill the functions of several items recited in the claims. The mere fact that certain measures are recited in mutually different dependent claims does not indicate that a combination of these measures cannot be used to advantage.

Any reference signs in the claims should not be construed as limiting the scope.

1. An elongated interventional device, configured to receive an optical shape sensing fiber and comprising:
an elongated proximal section including at fixation element at its proximal end, the fixation element being connectable to a reception at a predetermined location; and
an elongated distal section connected to the proximal section,
wherein the proximal section has a lower torsion stiffness than the distal section and wherein the proximal section comprises at least two substantially co-axial tubes, the outer tube having a lower torsion stiffness than an inner tube.

2. The elongated interventional device as defined in claim 1, wherein the proximal section and/or the distal section have a substantially homogenous torsion stiffness.

3. The elongated interventional device as defined in claim 1, wherein the inner tube has a higher kink resistance than the outer tube.

4. The elongated interventional device as defined in claim 1, wherein only the inner tube comprises at least one metal wire.

5. The elongated interventional device as defined in claim 4, wherein a plurality of metal wires form a braiding and/or wherein at least one metal wire forms a spiral.

6. The elongated interventional device as defined in claim 1, wherein the proximal section comprises three substantially co-axial tubes and wherein an inner tube has a lower friction coefficient than the other tubes.

7. The elongated interventional device as defined in claim 1, wherein the fixation element is configured to affix only the outer tube to the reception.

8. The elongated interventional device as defined in claim 7, wherein the inner tube can move relative to the outer tube of the proximal section.

9. The elongated interventional device 1 as defined in claim 1, comprising a catheter or guide wire.

10. An interventional system for performing an interventional procedure at a patient body, comprising:
an elongated interventional device as defined in claim 1 comprising the optical shape sensing fiber;
the reception arranged at the predetermined location and connectable with the fixation element of the elongated interventional device; and
an optical shape sensing device, the optical shape sensing device being coupled to the optical shape sensing fiber and being configured to determine a shape of the optical fiber and a position of the optical fiber relative to the predetermined location by optical shape sensing.

11. The interventional system as defined in claim 10, wherein the optical shape sensing device is configured to generate an image of the elongated interventional device on the basis of the determined shape and position.

12. The interventional system as defined in claim 11, wherein the optical shape sensing device is configured to overlay the image of the elongated interventional device and an image of the patient body, the image of the patient body being shown in accordance with a relative position of the patient body with respect to the predetermined location.

13. The interventional system as defined in claim 12, wherein the optical shape sensing device is configured to overlay the images in such a way that the relative position of the image of the elongated interventional device and the image of the patient body correspond to the relative position of the interventional device and the patient body.

14. The interventional system as defined in claim 11, wherein the image of the elongated interventional device and/or the image of the patient body are three-dimensional images.
